(19) United States
(12) Patent Application Publication
(10) Pub. No.: US 2023/0364386 A1
(43) Pub. Date: Nov. 16, 2023

(54) Systems, Medical Devices, and Methods for Steering the Medical Devices

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 17/742,098

(22) Filed: May 11, 2022

(51) Int. Cl. A61M 25/01

(52) U.S. Cl. CPC A61M 25/0144; A61B 2017/003

(57) ABSTRACT

Systems, medical devices, and methods for steering the medical devices are disclosed. For example, a system can include an elongate medical device and a pump station. The medical device can include a tubular body with a lumen. The lumen, which terminates proximal of a distal end of the medical device, can be configured to contain a fluid. The pump station can be configured to pressurize the fluid and, thereby, induce a curve in at least a distal portion of the medical device for steering the medical device through one or more anatomical lumens. Being as the tubular body of the medical device (e.g., a stylet) is configured to be disposed in another lumen of another elongate medical device (e.g., a catheter), any curvature in the medical device can be imparted to the other medical device when disposed therein for steering the other medical device through the one-or-more anatomical lumens.

### BACKGROUND

Current solutions aimed at steering elongate medical devices such as stylets typically lack real-time control. Indeed, the current solutions aimed at steering elongate medical devices are generally static. What is needed is real-time steering of elongate medical devices.

Disclosed herein are systems, medical devices, and methods that address the foregoing.

### SUMMARY

Disclosed herein is a system including, in some embodiments, an elongate medical device and a fluid-pressurizing means for pressurizing a fluid. The medical device includes a tubular body and a lumen therein. The lumen, which terminates proximal of a distal end of the medical device, is configured to contain the fluid therein. The fluid-pressurizing means is for pressurizing the fluid and, thereby, inducing a curve in at least a distal portion of the medical device under control of a user for steering the medical device through an anatomical lumen or a network of anatomical lumens.

In some embodiments, the fluid-pressurizing means is also for depressurizing the fluid and, thereby, softening or reducing the curve in at least the distal portion of the medical device under the control of the user for the steering of the medical device through the anatomical lumen or the network of anatomical lumens.

In some embodiments, the fluid-pressurizing means is a pneumatic or hydraulic pump station including a pump configured to pressurize and depressurize the fluid.

In some embodiments, the pump station is configured with a processor, memory, and associated logic configured to pressurize the fluid along a continuum for a corresponding continuum of curvature in at least the distal portion of the medical device under the control of the user.

In some embodiments, the fluid-pressurizing means is a syringe or bulb configured to pressurize and depressurize the fluid.

In some embodiments, the fluid is a gas.

In some embodiments, the fluid is a liquid.

In some embodiments, the lumen is offset from a central axis of the medical device. The lumen being offset from the central axis of the medical device allows tension along a side of the medical device including the lumen as well as compression along an opposite side of the medical device from the lumen when the fluid is pressurized for inducing the curve in at least the distal portion of the medical device.

In some embodiments, the medical device further includes a signal-conducting means for conducting a signal along a length of the medical device. The signal-conducting means is offset from the central axis of the medical device in the opposite side of the medical device from the lumen.

In some embodiments, the signal-conducting means is a wire configured to conduct electrical signals.

In some embodiments, the signal-conducting means is an optical fiber configured to conduct optical signals.

In some embodiments, the medical device is a stylet. The tubular body of the stylet is configured to be disposed in a lumen of another elongate medical device.

In some embodiments, the other elongate medical device is an intravenous catheter.

Also disclosed herein is another system including, in some embodiments, an elongate medical device and a fluid-pressurizing means for pressurizing a fluid. The medical device includes a tubular body and at least a pair of opposing membraned sacs over the tubular body. Each membraned sac of the pair of opposing membraned sacs, which membraned sac terminates at a distal end of the medical device, is configured to contain the fluid therein. The fluid-pressurizing means is for pressurizing the fluid in one or another membraned sac of the pair of membraned sacs and, thereby, inducing a curve in at least a distal portion of the medical device under control of a user for steering the medical device through an anatomical lumen or a network of anatomical lumens.

In some embodiments, the fluid-pressurizing means is also for depressurizing the fluid and, thereby, softening or reducing the curve in at least the distal portion of the medical device under the control of the user for the steering of the medical device through the anatomical lumen or the network of anatomical lumens.

In some embodiments, the fluid-pressurizing means is a pneumatic or hydraulic pump station including a pump configured to pressurize and depressurize the fluid.

In some embodiments, the pump station is configured with a processor, memory, and associated logic configured to pressurize the fluid along a continuum for a corresponding continuum of curvature in at least the distal portion of the medical device under the control of the user.

In some embodiments, the fluid-pressurizing means is a syringe or bulb configured to pressurize and depressurize the fluid.

In some embodiments, the fluid is a gas.

In some embodiments, the fluid is a liquid.

In some embodiments, the medical device further includes a signal-conducting means for conducting a signal along a length of the medical device.

In some embodiments, the signal-conducting means is a wire configured to conduct electrical signals.

In some embodiments, the signal-conducting means is an optical fiber configured to conduct optical signals.

In some embodiments, the medical device is a stylet. The tubular body of the stylet is configured to be disposed in a lumen of another elongate medical device.

In some embodiments, the other elongate medical device is an intravenous catheter.

Also disclosed herein is a method including, in some embodiments, a disposing step, a coupling step, a filling step, and a pressurizing step. The disposing step includes disposing a first elongate medical device in a second lumen of a second elongate medical device. The first medical device includes a tubular body and a first lumen therein. The first lumen, which terminates proximal of a distal end of the first medical device, is configured to contain a fluid therein. The coupling step includes coupling the first medical device to a fluid-pressurizing means for pressurizing the fluid. The filling step includes filling the first lumen of the first medical device with the fluid. The pressurizing step includes pressurizing the fluid in the first lumen of the first medical device, thereby, inducing a curve in at least a distal portion of the first medical device under control of a user for steering the medical device through an anatomical lumen or a network of anatomical lumens.

In some embodiments, the method further includes a depressurizing step. The depressurizing step includes depressurizing the fluid in the first lumen of the first medical device, thereby, softening or reducing the curve in at least the distal portion of the first medical device under the control of the user for the steering of the medical device through the anatomical lumen or the network of anatomical lumens.

In some embodiments, the fluid is a gas.

In some embodiments, the fluid is a liquid.

In some embodiments, the first lumen is offset from a central axis of the first medical device. The first lumen being offset from the central axis of the first medical device allows tension along a side of the first medical device including the first lumen as well as compression along an opposite side of the first medical device from the first lumen when the fluid is pressurized for the inducing of the curve in at least the distal portion of the first medical device.

In some embodiments, the first medical device is a stylet and the second medical device is an intravenous catheter.

These and other features of the concepts provided herein will become more apparent to those of skill in the art in view of the accompanying drawings and following description, which describe particular embodiments of such concepts in greater detail.

### DRAWINGS

FIG. 1 illustrates a system for controlling curvature of an elongate medical device such as a stylet in accordance with some embodiments.

FIG. 2 illustrates the stylet disposed in another elongate medical devices such as an intravenous catheter in accordance with some embodiments.

FIG. 3 illustrates a transverse cross section of the stylet disposed in the catheter in accordance with some embodiments.

FIG. 4 illustrates a perspective view of the stylet in accordance with some embodiments.

FIG. 5 illustrates a transition of the stylet from an unpressurized state to a pressurized state with an induced curve on account of a fluid-pressurized lumen in accordance with some embodiments.

FIG. 6 illustrates a transverse cross section of the stylet of FIG. 5 in the unpressurized state in accordance with some embodiments.

FIG. 7 illustrates a transverse cross section of the stylet of FIG. 5 in the pressurized state in accordance with some embodiments.

FIG. 8 illustrates a transition of the stylet from an unpressurized state to a pressurized state with an induced curve on account of a fluid-pressurized membraned sac in accordance with some embodiments.

FIG. 9 illustrates a detailed view of the stylet of FIG. 8 in the unpressurized state in accordance with some embodiments.

FIG. 10 illustrates a detailed view of the stylet of FIG. 8 in the pressurized state in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a stylet includes a portion or section of the stylet intended to be near a clinician when the stylet is used on a patient. Likewise, a “proximal length” of, for example, the stylet includes a length of the stylet intended to be near the clinician when the stylet is used on the patient. A “proximal end” of, for example, the stylet includes an end of the stylet intended to be near the clinician when the stylet is used on the patient. The proximal portion, the proximal section, or the proximal length of the stylet can include the proximal end of the stylet; however, the proximal portion, the proximal section, or the proximal length of the stylet need not include the proximal end of the stylet. That is, unless context suggests otherwise, the proximal portion, the proximal section, or the proximal length of the stylet is not a terminal portion or terminal length of the stylet.

With respect to “distal,” a “distal portion” or a “distal section” of, for example, a stylet includes a portion or section of the stylet intended to be near or in a patient when the stylet is used on the patient. Likewise, a “distal length” of, for example, the stylet includes a length of the stylet intended to be near or in the patient when the stylet is used on the patient. A “distal end” of, for example, the stylet includes an end of the stylet intended to be near or in the patient when the stylet is used on the patient. The distal portion, the distal section, or the distal length of the stylet can include the distal end of the stylet; however, the distal portion, the distal section, or the distal length of the stylet need not include the distal end of the stylet. That is, unless context suggests otherwise, the distal portion, the distal section, or the distal length of the stylet is not a terminal portion or terminal length of the stylet.

Unless defined otherwise, all technical and scientific terms used herein have the same meaning as commonly understood by those of ordinary skill in the art.

As set forth above, the current solutions aimed at steering elongate medical devices such as stylets typically lack real-time control. Indeed, the current solutions aimed at steering elongate medical devices are generally static. What is needed is real-time steering of elongate medical devices.

Disclosed herein are systems, medical devices, and methods for steering the medical devices. For example, a system can include an elongate medical device such as a stylet and a fluid-pressurizing means such as a pneumatic or hydraulic pump station for pressurizing a fluid. The medical device can include a tubular body with a lumen. The lumen, which terminates proximal of a distal end of the medical device, can be configured to contain the fluid. The fluid-pressurizing means is for pressurizing the fluid and, thereby, inducing a curve in at least a distal portion of the medical device under control of a user for steering the medical device through an anatomical lumen or a network of anatomical lumens. Being as the tubular body of the medical device is configured to be disposed in another lumen of another elongate medical device such as an intravenous catheter, any curvature in the medical device (e.g., the stylet) can be imparted to the other medical device (e.g., the catheter) when disposed therein for steering the other medical device through the anatomical lumen or the network of anatomical lumens.

### Systems

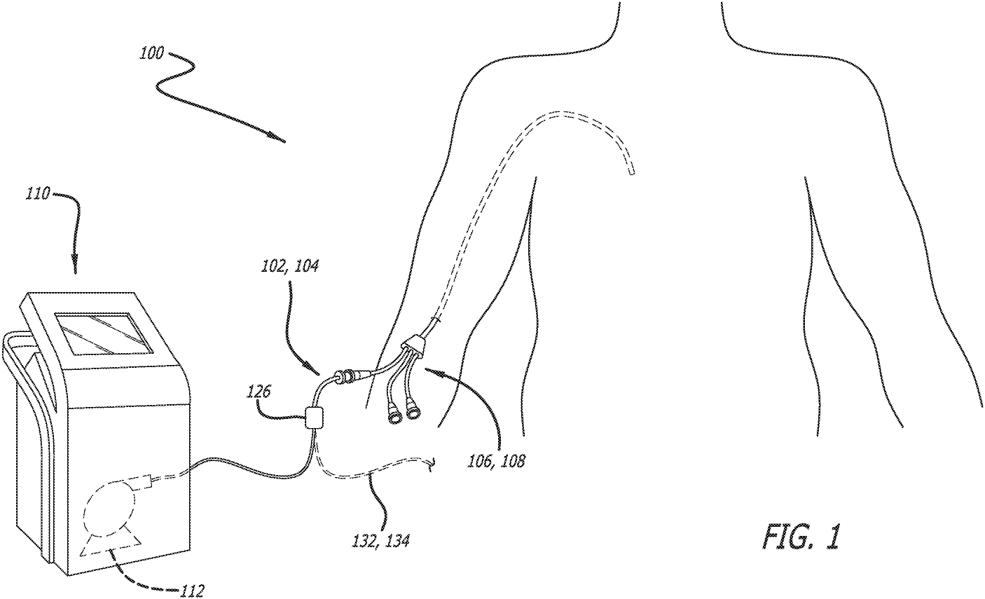

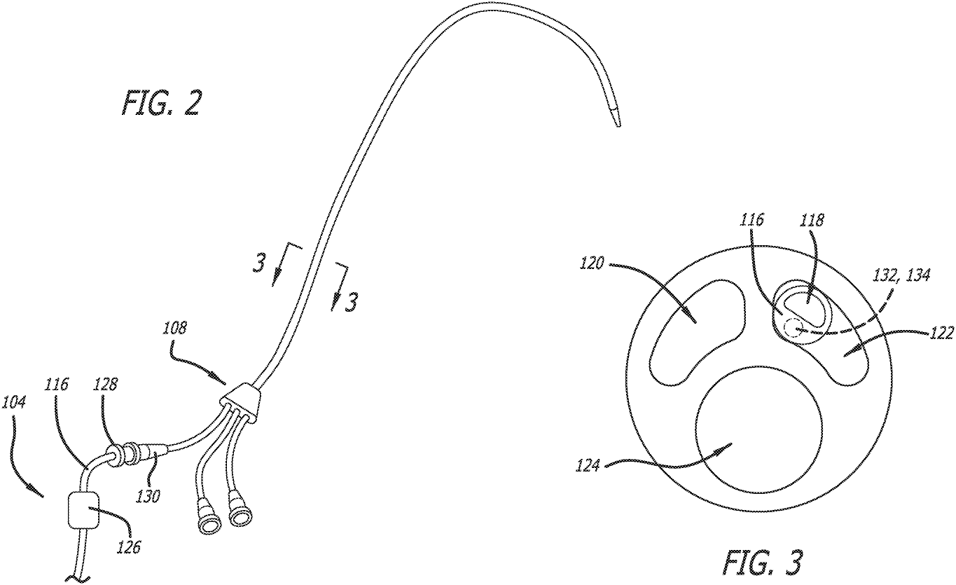

FIG. 1 illustrates a system 100 for steering an elongate medical device 102 such as a stylet 104 in accordance with some embodiments. FIG. 2 illustrates the stylet 104 disposed in another elongate medical device 106 such as an intravenous catheter 108 in accordance with some embodiments. FIG. 3 illustrates a transverse cross section of the stylet 104 disposed in the catheter 108 in accordance with some embodiments.

As shown, the system 100 can include a pneumatic or hydraulic pump station 110 and at least the medical device 102, for example, the stylet 104. Notably, the system 100 can also include the other medical device 106, for example, the catheter 108, into which the medical device 102 is configured to be disposed. Indeed, the tubular body 116 of the medical device 102 is configured to be disposed in the other medical device 106 to control curvature in the other medical device 106 by controlling the curvature in the medical device 102. And, by controlling the curvature in the medical device 102, the other medical device 106 can be steered through, for example, an anatomical lumen or a network of anatomical lumens (e.g., a vasculature).

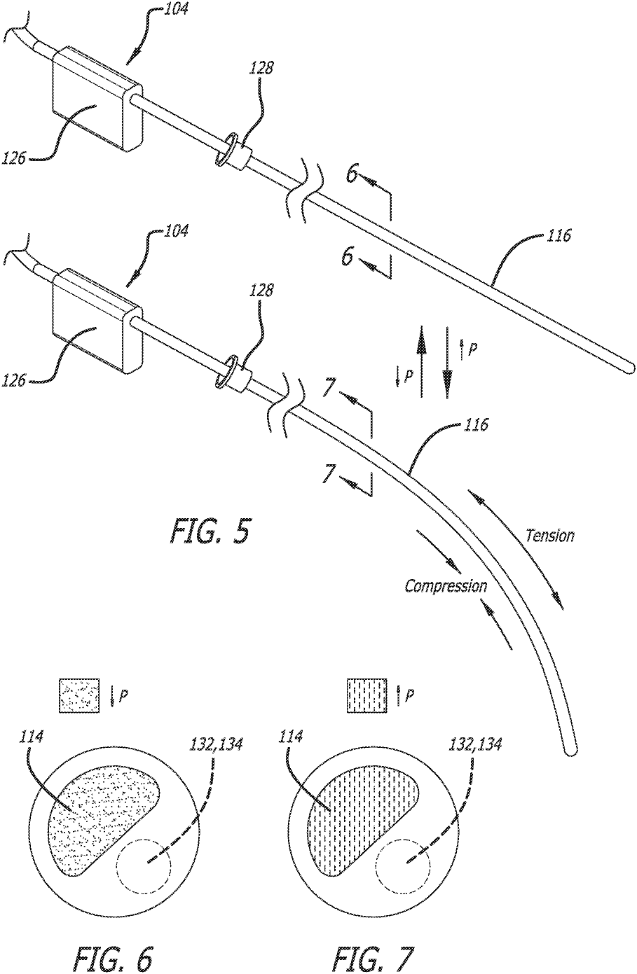

When present, the pump station 110 includes a pump 112 and an embedded system (not shown) for operating the system 100 under control of a user such as a clinician. The pump 112 is configured to pressurize a fluid 114 in the medical device 102 and, thereby, induce a curve in at least a distal portion of the medical device 102 for steering the medical device 102 through, for example, the anatomical lumen or the network of anatomical lumens. The pump 112 is also configured to depressurize the fluid 114 and, thereby, soften or reduce the curve in at least the distal portion of the medical device 102 for the steering of the medical device 102 through, for example, the anatomical lumen or the network of anatomical lumens. (See, for example, FIG. 5.) The embedded system can include a system on a chip, a microcontroller, or the like having a processor, memory, and associated logic configured to pressurize the fluid 114 along a continuum for a corresponding continuum of curvature in at least the distal portion of the medical device 102. Likewise, the embedded system is configured to depressurize the fluid 114 along the continuum for the corresponding continuum of curvature in at least the distal portion of the medical device 102.

Notably, whether the pump station 110 is present or absent, the fluid-pressurizing means can alternatively be a syringe, a bulb (e.g., a pipet bulb), or some other hand-operated pump-like device configured to pressurize and depressurize the fluid 114 under control of the user.

The fluid 114 for which the system 100 is configured can include a gas such as nitrogen, argon, or compressed air or a liquid such as water or saline. Such a fluid can be stored in a reservoir such as a replaceable tank or canister fluidly connected to the pump 112.

### Medical Devices

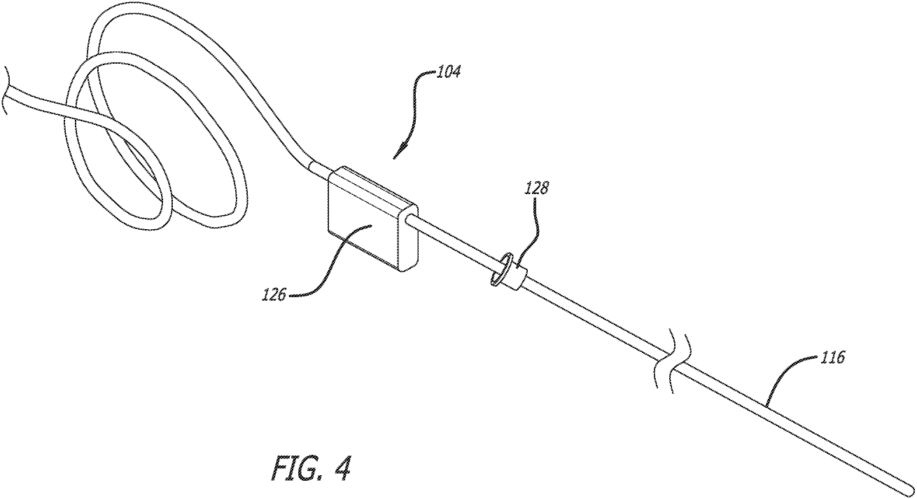

FIG. 4 illustrates a perspective view of the stylet 104 in accordance with some embodiments.

As shown, the medical device 102 can be the stylet 104. For convenience, the stylet 104 will serve as a particular species of the medical device 102 herein. However, it should be understood that the medical device 102, being the genus to which the stylet 104 belongs, includes features of the stylet 104 set forth below.

The stylet 104 can include a tubular body 116 and a lumen 118 in the tubular body 116 in some embodiments. However, in other embodiments, the stylet 104 can include at least a pair of opposing membraned sacs 136 over the tubular body 116 in addition to or as an alternative of the lumen 118.

The tubular body 116 of the stylet 104 is configured to be disposed in a lumen of the catheter 108 as a species of the other medical device 106 to control curvature in the catheter 108 by controlling the curvature in the stylet 104. Indeed, FIGS. 2 and 3 show the stylet 104 disposed in a secondary or tertiary lumen 120 or 122 of the catheter 108 for controlling the curvature in the catheter 108. However, the stylet 104 can be disposed in a primary lumen 124 of the catheter 108 for controlling the curvature in the catheter 108 all the way to a distal tip of the catheter 108, which distal tip the secondary or tertiary lumen 120 or 122 are typically short of. By controlling the curvature in the catheter 108 as a species of the medical device 106, the catheter 108 can be steered through, for example, the anatomical lumen or the network of anatomical lumens (e.g., a vasculature). Notably, the tubular body 116 of the stylet 104 can be formed of a polymer (e.g., a polyurethane), optionally, in a multilayered structure including an intervening reinforcement layer for higher pressures. While not shown, such an intervening reinforcement layer can be a braided tube of a metal or a same or different polymer than a remainder of the tubular body 116.

FIG. 5 illustrates a transition of the stylet 104 from an unpressurized state to a pressurized state with an induced curve on account of the fluid 114 pressurized in the lumen 118 in accordance with some embodiments. FIG. 6 illustrates a transverse cross section of the stylet 104 in the unpressurized state in accordance with some embodiments. FIG. 7 illustrates a transverse cross section of the stylet 104 in the pressurized state in accordance with some embodiments.

As shown, the lumen 118, which terminates proximal of a distal end of the stylet 104, is configured to contain the fluid 114 therein. The fluid 114, when pressurized, induces a curve in at least a distal portion of the stylet 104 under control of the user for steering the stylet 104 through, for example, the anatomical lumen or the network of anatomical lumens. Notably, the lumen 118 is offset from a central axis of the stylet 104. The lumen 118 being offset from the central axis of the stylet 104 allows tension along a side of the stylet 104 including the lumen 118 as well as compression along an opposite side of the stylet 104 from the lumen 118 when the fluid 114 is pressurized for inducing the curve in at least the distal portion of the stylet 104.

As an alternative to the lumen 118, the stylet 104 can include a channel in a side of the tubular body 116. While not shown, the channel, when present, can form a temporary lumen between it and a luminal wall of the catheter 108 when the stylet 104 having the channel is disposed a lumen of the catheter 108. Like the lumen 118 set forth above, the temporary lumen formed from the channel is offset from the central axis of the stylet 104, which allows tension along a side of the catheter 108 over the channel as well as compression along an opposite side of the stylet 104 from the channel when the fluid 114 is pressurized for inducing the curve in at least the distal portion of the stylet 104.

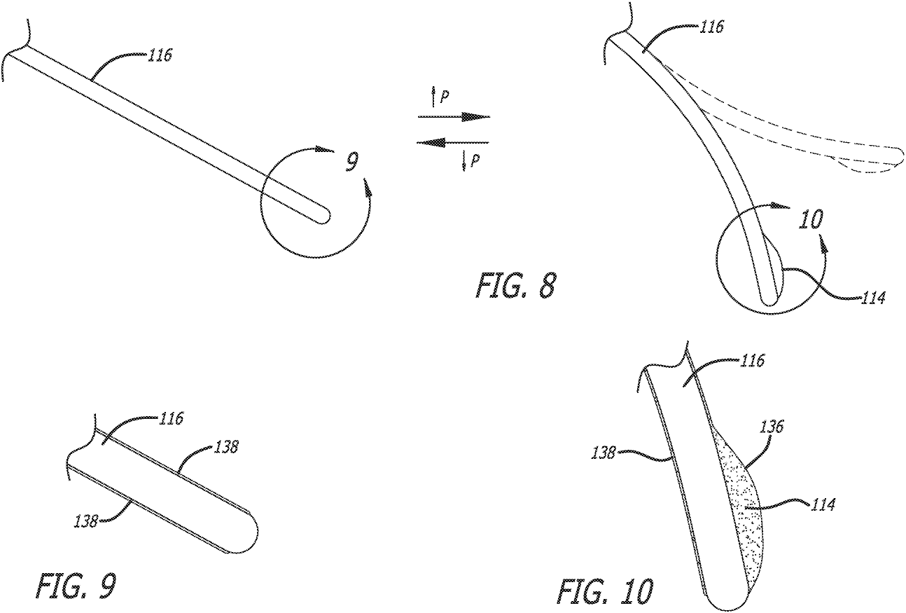

FIG. 8 illustrates another transition of the stylet 104 from an unpressurized state to a pressurized state with an induced curve on account of the fluid 114 pressurized in a membraned sac 136 of the pair of membraned sacs 136 in accordance with some embodiments. FIG. 9 illustrates a detailed view of the stylet 104 of FIG. 8 in the unpressurized state in accordance with some embodiments. FIG. 10 illustrates a detailed view of the stylet 104 of FIG. 8 in the pressurized state in accordance with some embodiments.

As shown, each membraned sac 136 of the pair of opposing membraned sacs 136, which membraned sacs 136 terminate at the distal end of the stylet 104, is configured to contain the fluid 114 therein. The fluid 114, when pressurized in one or another membraned sac 136 of the pair of membraned sacs 136, induces a curve in at least the distal portion of the stylet 104 under control of the user for steering the stylet 104 through, for example, the anatomical lumen or the network of anatomical lumens. Notably, the stylet 104 is shown with the pair of opposing membraned sacs 136. When the fluid 114 is pressurized in the one or the other membraned sac 136 of the pair of membraned sacs 136 and the curve is induced in at least the distal portion of the stylet 104, the curve curves away from the membraned sac 136 being pressurized. It should be understood the stylet 104 is not limited to the pair of opposing membraned sacs 136. Indeed, each additional membraned sac 136 provides an additional direction in which the stylet 104 can be curved for steering the stylet 104, where three membraned sacs 136 in the distal portion of the stylet 104 provide three different directions in which the stylet 104 can be steered, four membraned sacs 136 in the distal portion of the stylet 104 provide four different directions in which the stylet 104 can be steered, and so on. Lastly, the lumen 118 of the stylet 104 can be divided into a number of sac-supply lumens by a corresponding number of longitudinal septa to provide the fluid 114 to a number of the membraned sacs 136 over the tubular body 116 of the stylet 104. Alternatively, as shown, a number of sac-supply conduits 138 over the tubular body 116 of the stylet 104 can provide the fluid 114 to the number of the membraned sacs 136.

The stylet 104 can further include a handle 126 around the tubular body 116 of the stylet 104 but proximal of an insertable portion of the tubular body 116. The handle 126, when present, is configured for handling the stylet 104 thereby.

The stylet 104 can further include a Luer connector 128 around the tubular body 116 of the stylet 104 configured to insert or screw into a complementary Luer connector 130 of the catheter 108. The Luer connector 128 can be slidably disposed around the tubular body 116 of the stylet 104 for adjusting a length of the stylet 104 disposed in the catheter 108.

The stylet 104 can further include a signal-conducting means for conducting a signal along a length of the medical device 102. For example, the signal-conducting means can be a wire 132 configured to conduct electrical signals such as ECG signals, or the signal-conducting means can be an optical fiber 134 configured to conduct optical signals such as those reflected back from fiber Bragg gratings (“FBGs”) in the optical fiber 134. Such a signal-conducting means, when present, can be offset from the central axis of the stylet 104 in the opposite side of the stylet 104 from the lumen 118 as shown in FIGS. 3, 6, and 7. However, the signal-conducting means, when present, can alternatively be aligned with the central axis of the stylet 104 when, for example, the stylet 104 includes the membraned sacs 136.

### Methods

Methods include at least a method of using the system 100, for example, to steer the catheter 108. Such a method can include one or more steps selected from a disposing step, a coupling step, a filling step, a pressurizing step, and a depressurizing step.

The disposing step includes disposing the stylet 104 in a lumen (e.g., the primary, secondary, or tertiary lumen 120, 122, or 124) of the catheter 108. Again, the stylet 104 includes the tubular body 116 and, optionally, the lumen 118 in the tubular body 116, the pair of membraned sacs 136 over the tubular body 116, or both.

The coupling step includes coupling the stylet 104 to the fluid-pressurizing means for pressurizing the fluid 114. Again, the fluid-pressurizing means for pressurizing the fluid 114 can be the pneumatic or hydraulic pump station 110 or some other hand-operated pump-like device configured to pressurize and depressurize the fluid 114 under control of the user.

The filling step includes filling the lumen 118 of the stylet 104, the pair of membraned sacs 136 of the stylet 104, or both with the fluid 114.

The pressurizing step includes pressurizing the fluid 114 in the lumen 118 or the pair of membraned sacs 136 of the stylet 104, thereby, inducing the curve in at least the distal portion of the stylet 104 under control of the user for steering the stylet 104 through, for example, the anatomical lumen or the network of anatomical lumens. Optionally, the steering of the stylet 104 can be under guidance of a strain-sensing subsystem using the optical fiber 134.

The depressurizing step includes depressurizing the fluid 114 in the lumen 118 of the stylet 104, thereby, softening or reducing the curve in at least the distal portion of the stylet 104 under control of the user for the steering of the stylet 104 through, for example, the anatomical lumen or the network of anatomical lumens. Again, the steering of the stylet 104 can be under guidance of a strain-sensing subsystem using the optical fiber 134.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A system, comprising:
an elongate medical device including:
a tubular body; and
a lumen terminating proximal of a distal end of the medical device, the lumen configured to contain a fluid therein; and
a fluid-pressurizing means for pressurizing the fluid and, thereby, inducing a curve in at least a distal portion of the medical device under control of a user for steering the medical device through an anatomical lumen or a network of anatomical lumens.

2. The system of claim 1, wherein the fluid-pressurizing means is also for depressurizing the fluid and, thereby, softening or reducing the curve in at least the distal portion of the medical device under the control of the user for the steering of the medical device through the anatomical lumen or the network of anatomical lumens.

3. The system of claim 2, wherein the fluid-pressurizing means is a pneumatic or hydraulic pump station including a pump configured to pressurize and depressurize the fluid.

4. The system of claim 3, wherein the pump station is configured with a processor, memory, and associated logic configured to pressurize the fluid along a continuum for a corresponding continuum of curvature in at least the distal portion of the medical device under the control of the user.

5. The system of claim 2, wherein the fluid-pressurizing means is a syringe or bulb configured to pressurize and depressurize the fluid.

6. The system of claim 1, wherein the fluid is a gas.

7. The system of claim 1, wherein the fluid is a liquid.

8. The system of claim 1, wherein the lumen is offset from a central axis of the medical device to allow tension along a side of the medical device including the lumen and compression along an opposite side of the medical device from the lumen when the fluid is pressurized for inducing the curve in at least the distal portion of the medical device.

9. The system of claim 8, wherein the medical device further includes a signal-conducting means for conducting a signal along a length of the medical device, the signal-conducting means offset from the central axis of the medical device in the opposite side of the medical device from the lumen.

10. The system of claim 9, wherein the signal-conducting means is a wire configured to conduct electrical signals.

11. The system of claim 9, wherein the signal-conducting means is an optical fiber configured to conduct optical signals.

12. The system of claim 1, wherein the medical device is a stylet, the tubular body of the stylet configured to be disposed in a lumen of another elongate medical device.

13. The system of claim 12, wherein the other elongate medical device is an intravenous catheter.

14. A system, comprising:
an elongate medical device including:
a tubular body; and
at least a pair of opposing membraned sacs over the tubular body terminating at a distal end of the medical device, each membraned sac of the pair of membraned sacs configured to contain a fluid therein; and
a fluid-pressurizing means for pressurizing the fluid in one or another membraned sac of the pair of membraned sacs and, thereby, inducing a curve in at least a distal portion of the medical device under control of a user for steering the medical device through an anatomical lumen or a network of anatomical lumens.

15. The system of claim 14, wherein the fluid-pressurizing means is also for depressurizing the fluid and, thereby, softening or reducing the curve in at least the distal portion of the medical device under the control of the user for the steering of the medical device through the anatomical lumen or the network of anatomical lumens.

16. The system of claim 15, wherein the fluid-pressurizing means is a pneumatic or hydraulic pump station including a pump configured to pressurize and depressurize the fluid.

17. The system of claim 16, wherein the pump station is configured with a processor, memory, and associated logic configured to pressurize the fluid along a continuum for a corresponding continuum of curvature in at least the distal portion of the medical device under the control of the user.

18. The system of claim 15, wherein the fluid-pressurizing means is a syringe or bulb configured to pressurize and depressurize the fluid.

19. The system of claim 14, wherein the fluid is a gas.

20. The system of claim 14, wherein the fluid is a liquid.

21. The system of claim 14, wherein the medical device further includes a signal-conducting means for conducting a signal along a length of the medical device.

22. The system of claim 21, wherein the signal-conducting means is a wire configured to conduct electrical signals.

23. The system of claim 21, wherein the signal-conducting means is an optical fiber configured to conduct optical signals.

24. The system of claim 14, wherein the medical device is a stylet, the tubular body of the stylet configured to be disposed in a lumen of another elongate medical device.

25. The system of claim 24, wherein the other elongate medical device is an intravenous catheter.

26. A method, comprising:
disposing a first elongate medical device in a second lumen of a second elongate medical device, the first medical device including:
a tubular body; and
a first lumen terminating proximal of a distal end of the first medical device, the first lumen configured to contain a fluid therein; and
coupling the first medical device to a fluid-pressurizing means for pressurizing the fluid;
filling the first lumen of the first medical device with the fluid; and
pressurizing the fluid in the first lumen of the first medical device, thereby, inducing a curve in at least a distal portion of the first medical device under control of a user for steering the medical device through an anatomical lumen or a network of anatomical lumens.

27. The method of claim 26, further comprising:
depressurizing the fluid in the first lumen of the first medical device, thereby, softening or reducing the curve in at least the distal portion of the first medical device under the control of the user for the steering of the medical device through the anatomical lumen or the network of anatomical lumens.

28. The method of claim 27, wherein the fluid is a gas.

29. The method of claim 27, wherein the fluid is a liquid.

30. The method of claim 27, wherein the first lumen is offset from a central axis of the first medical device to allow tension along a side of the first medical device including the first lumen and compression along an opposite side of the first medical device from the first lumen when the fluid is pressurized for the inducing of the curve in at least the distal portion of the first medical device.

31. The method of claim 27, wherein the first medical device is a stylet and the second medical device is an intravenous catheter.
